(19) United States
(12) Patent Application Publication
(10) Pub. No.: US 2014/0163599 A1
(43) Pub. Date: Jun. 12, 2014

(54) Lumen Occluding Stent, Delivery Catheter and Method

(71) Applicant: Bandula Wijay, Friendswood, TX (US)

(72) Inventor: Bandula Wijay, Friendswood, TX (US)

(21) Appl. No.: 13/707,272

(22) Filed: Dec. 6, 2012

(51) Int. Cl. A61B 17/12

(52) U.S. Cl. CPC A61B 17/12
USPC 606/192

(57) ABSTRACT

A permanent acute occlusion implantable device and method are described for immediate occlusion of a body lumen, such as blood vessels and the fallopian tubes of the human female, wherein an balloon expandable lumen occlusion device is placed within the lumen to be occluded. After inflating the balloon, the balloon is withdrawn leaving the expanded occlusion device in the lumen. The device is encased in an occluding impervious sheath and therefore completely occludes the lumen once the device is delivered and anchored into the inner wall of the body lumen.

### FIELD OF THE INVENTION

The present invention relates to an endo-luminal occluding device, which when implanted within a body lumen, such as arteries, veins, fallopian tubes, facilitates immediate occlusion of the lumen. By this means blood supply to tumors, necrotic tissues, and aneurisms can be cut off immediately and help in healing of the affected areas of interest. Similarly an immediate blocking of the fallopian tubes will prevent sperm migration preventing the fertilization of the ovum and hence will provide means of immediate female sterilization.

### BACKGROUND OF THE INVENTION

The present invention deals with a method and device that will occlude the body lumen immediately and does not depend of tissue growth to cause the occlusion of the body lumen. Occlusion of body lumens for various medical procedures as well as for female sterilization is a very common practice and is performed frequently throughout the world except in most cases the procedure depends of tissue growth due to the tissue injury to affect closure, which takes time. Often additional tests are conducted at a later date, with additional expenditure, to assure, a closed lumen. Cutting blood supply to tumors is often used to shrink the tumor and similarly cutting blood supply can also alleviate neuropathic pain in the clinical practice. For female sterilization, various devices have been developed in the past and these methods depend on tissue growth within the lumen caused by injury and/or irritation of the fallopian tube tissue by an implanted device for its closure. Additionally, the most common female sterilization method is fallopian tube ligation, a procedure that utilizes a trans-abdominal approach for the occlusion, or tying, of the fallopian tubes, which is surgical, and expensive. Despite its worldwide use, tubal ligation via the trans-abdominal approach is associated with substantial trauma, discomfort, hospital stays, and complications, such as bleeding, infection, reactions to general anesthetic, and bowel perforation. The trans-abdominal approach involves surgery, and is not readily available to many women throughout the world. Even though local anesthetic is considered an option for the trans-abdominal approach to tubal ligation, almost all of these sterilization procedures are performed under general or spinal anesthesia. In addition, the trans-abdominal approach to tubal ligation requires incisions that invade the peritoneal cavity, thereby raising the risk of injury to intra-abdominal structures.

In order to avoid the problems associated with trans-abdominal tubal ligation procedures, various trans-cervical approaches to tubal sterilization have been proposed. The trans-cervical approach to sterilization involves the insertion of a catheter or sterilization device directly into the fallopian tubes via the reproductive tract, eliminating the need for general anesthetic and abdominal incisions. Initial trans-cervical approaches to tubal sterilization involved radiofrequency, chemical or heat induced scarring, or liquid silicone injections. However, these approaches have all failed due to safety and efficacy concerns. Chemical scarring agents, such as quinacrine, iodine, and methylcyanoacrylate, require repeated applications and have problems concerning biocompatibility. Thermal blocking procedures, which induce the formation of scar tissue within the fallopian tubes, have high failure rates and major complications such as uterine bleeding and bowel perforation. Electrocautery methods, which employ an electric current to induce scar tissue within the fallopian tubes, are also unsatisfactory because they do nor scar a sufficient amount of tissue and because they can burn surrounding organs, particularly the bowel.

Current trans-cervical methods involve occluding the fallopian tubes by implanting a small occluding device. The occluding devices in the prior art are usually in the form of a cylindrical plug or a coil. For instance, Loy in U.S. Pat. No. 6,357,443 describes a removable fallopian tube plug consisting of a tubular (cylindrical) elongate member with a number of fingerlike protrusions that extend radially outwards creating a barrier and thereby occluding the fallopian tubes. Additionally, Saadat et al. in U.S. Pat. No. 5,935,137 describe a fallopian tube occluding device for female sterilization which is a plastic, rubber, or metal elongate hollow tubular (cylindrical) structure with ribs that are either coated with copper or are interlaced with copper rings. The hollow portion of this device has a valve, or seals with a hydrogel, after the device is implanted into the fallopian tubes.

Coils, which have a helical outer surface and which assume a bent shape when released from the delivery catheter system, are also used to occlude the fallopian tubes. For example, Ton et al. in U.S. Pat. Nos. 5,601,600 and 5,746,769 describe the use of a coil to occlude the fallopian tubes. The device consists of polyethylene terephthalate (PET) fibers wrapped around a stainless steel core that is surrounded by 24 or more coils of nickel-titanium alloy. After the device is deployed within the fallopian tubes, the PET fibers induce the tubal epithelium to undergo fibrosis, which results in tubal occlusion. The device also relaxes to its natural bent shape once it is deployed in the fallopian tube. The tubal occlusion process from these devices takes about three months to complete and must be confirmed via a hysterosalpingogram.

U.S. Pat. No. 7,846,160 teaches the use of an exterior screw thread to advance a lumen plug by rotation In FIG. 3. In FIG. 4 barbs are held under a sheath such that when the sheath is retracted, the barbs spring out and point proximally to prevent removal.

Most of these devices eventually become dislodged or have found to be only moderately effective in preventing pregnancy. In addition, all of the fallopian tube occluding devices are either composed of metal or have metal components. As a result, various surgical procedures involving electrosurgery, radiofrequency, or microwave energy cannot be performed near the implants. Therefore, a need exists for a female contraceptive device that does not contain any metal, does not migrate once implanted, and which provides immediate protection against conception. The occlusion device of the present invention fulfills these needs.

### SUMMARY OF THE INVENTION

The present invention consists of a permanent acute occlusion implantable device and method for immediate occlusion of a body lumen, such as the blood vessels, aneurisms, or the fallopian tubes of the human female. The implant, an acute occlusion device, consists of a small narrow low profile slotted tube (stent) and which carries a hemi-spherical or conical-shaped structure, which is a continuation of the struts of the slotted tube, that is located near the distal end of the small narrow slotted tube, the struts of which are at least in part encased in an expandable sheath, which can function as an occlusion means for a body lumen when it is expanded using an inflatable balloon.

The occlusion means described in the present invention is permanently placed inside a body lumen for occlusion of the body lumen. The device is delivered using a balloon where the occlusion element is crimped onto a balloon. The delivery catheter is threaded into the appropriate location either through a sheath or through a hysteroscope and the balloon is inflated to deliver the occlusion device into place causing immediate occlusion of the lumen.

The small narrow slotted tube (or the stent) is made from a metallic tube by cutting appropriate slots, or openings, on its surface, so that expanding of the slotted tube using an inflating balloon as previously described, yields a structure that is several times larger than its original diameter and continues to remain at its final expanded diameter, depending on the diameter of the inflating balloon. The material of the slotted tube (stent) will undergo deformation beyond its yield point and therefore will remain at or near its expanded condition. Often, a slightly higher inflating diameter may be required to achieve the final diameter needed for the expanded slotted tube for a given application. The initial diameter of the slotted tube is substantially small (less than 2 mm) so that the initial profile of the slotted tube and the entire device is small enough to thread through a delivery device catheter or a hysteroscope depending on the purpose of its use. The slots or openings on the surface of the slotted tube can be lengthwise or at an angle to its longitudinal axis. The number of slots around its diameter also depends on the surface available on the slotted tube for providing sufficient slots. The slots can be arranged in many different configurations having the general sinusoidal or triangular undulations or any other shape that would make the slotted tube flexible. The width and the length of the slots are determined by the initial diameter, hence the surface area, of the slotted tube and the ratio of the expanded and the non expanded diameter of the slotted tube.

The slotted tube is mounted on a balloon by crimping on to the deflated (and folded) balloon typically by using a crimping means available for this purpose in the industry. The balloon material chosen shall be such that the balloon inflates to a pre-determined fixed diameter at a given inflation pressure. Typically a balloon made from nylon, PET or similar material is suitable for this purpose. The balloon catheter will have a relatively small shaft such as 2 French and will have sufficient stiffness and flexibility that allows optimal push-ability and track-ability characteristics.

The slotted tube or stent is encased in a bio-compatible expandable material, such as silicone, polyurethane or any other soft, expandable polymeric or biological material that can be used to encase the stent and the conical structure at the distal end of the occluding device. The encasing material shall be well bonded to the metallic struts of the slotted tube and the struts of its cone and shall not separate from the struts during expansion by the inflating balloon.

The occlusion device is provided with one or more protrusions, arising from the undulating segments, which are placed on the slotted tube body, which upon expansion will embed into the tissue to anchor the occlusion device into the inner wall of the occluding lumen. These protrusions can be placed anywhere along the largest diameter section of the occluding device.

Additionally the inflating balloon can be so made having a waist region in the middle area such that when the slotted tube is expanded, the slotted tube takes the shape of the balloon into a dumb-bell appearance. This feature allows the slotted tube to physically lock into the lumen tissue and will prevent the migration of the occluding device within the occluding lumen.

Another means to anchor would be to use an inflating balloon which has a larger diameter at its proximal end than the distal end. Thereby the expanded stent will have a larger diameter proximally than distally. When properly placed, the expanded portion of the slotted tube, which is larger than the rest of the slotted tube can be located at the ostium of the lumen, thereby preventing the occluding device migrating further deep into the occluding lumen.

The slotted tube generally has several undulating or sinusoidal segments that are connected with flexible connecting strut members. The connecting strut members can be straight, parabolic or sinusoidal and may have more than one tangent thereby creating a curved strut. The connecting struts can be very short (less than 0.5 mm) or long (more than 0.5 mm). The preferred embodiment will consist of two undulating rings in the slotted tube that is connected by a strut which is 3-5 mm long. The distal undulating ring continues to form a conical or hemispherical structure, which may be attached to a short guide wire at its distal end. The guide wire helps the track-ability of the device, in the event the lumen has complex and multiple curvatures or when the lumen takes off at an angle at the ostium. Typically the distal undulating segment and the conical portion are encased in the expandable membrane, which is impervious to body fluids, such as blood or semen.

Once the occluding device is properly delivered, a hermetic seal is made between the distal undulating segment of the occluding device and the inner wall of the lumen preventing fluid passage. The cone portion having encased struts is also completely impervious to any fluids and therefore no fluid passes through the cone section of the occluding device. The space between the two undulating segments is connected by a few struts (typically four) providing the special benefit in anchoring the device in the lumen as the inner wall of the lumen as the tissue will prolapse through the struts affixing the occlusion device in the lumen.

The encasing materials, such as silicone usually have elastic properties. As such when the slotted tube is expanded, there is some degree of recoil (back) of the slotted tube, which may cause an improper hermetic seal. Therefore the slotted tube can be designed to have two different diameters with one portion overlapping the other. This will allow less elastic deformation of the encasing material and therefore less recoil of the stent after delivery. This is accomplished by using a crimping technique whereby the struts at 12 o'clock and 6 o'clock are crimped first on the balloon and the struts orthogonal to them, i.e. the struts at 3 and 9 o'clock are crimped over the struts that were previously crimped. During inflation of the balloon, the encased slotted tube first unfolds and then expands slightly to its maximum desired dimension. This low expansion will reduce the recoil and help in a more reliable sealing of the slotted tube into the inner wall of the lumen.

The above described device is a balloon delivered occlusion device, which is made from a slotted tube encased with an expanding, yet impervious membrane. Upon delivery and the occluding device is anchored in the lumen and provides an occluding “wall” completely closing the lumen and preventing the flow of fluids across the device. As such, this device and various modification of it will provide permanent occlusion of a body lumen when properly implanted in a body lumen. The advantage of the present invention is that immediate hermetic seal and a complete occlusion of the lumen are achieved by the proper placement of the lumen occluding device. This device does not depend on tissue growth to close the lumen, which not only takes time but in some instances not very reliable and does not require a test after a few weeks to check for the efficacy of occlusion.

### BRIEF DESCRIPTION OF THE DRAWINGS

FIG. 1 shows the lumen occluding stent device.

FIG. 2 shows the lumen occluding stent device mounted on to a delivery catheter.

FIG. 3 shows a delivery catheter balloon (inflated) having a waist region, on which a lumen occluding stent device can be mounted.

FIG. 4 shows a cross section of an alternate way to fold and crimp a lumen occluding stent on to a balloon.

FIG. 5 shows the lumen occluding stent device with the balloon inflated to the desired diameter.

FIG. 6 shows the lumen occluding stent device mounted onto a balloon with a waist with the balloon inflated showing the form of the lumen occluding stent device, after balloon inflation.

FIG. 7 shows a lumen occluding stent device mounted onto a balloon having a larger diameter at the balloon's proximal end, and the stent expanded taking the shape of the balloon.

FIG. 8 shows a lumen occluding stent having a proximal section expanded to a larger diameter than the rest of the stent using a balloon having a larger proximal portion.

FIG. 9 shows the lumen occluding stent device placed in the ostium of the fallopian tube of the human female.

FIG. 10 shows the lumen occluding stent device having only two segments connected by long struts placed in the ostium of the fallopian tubes of the human female, also showing the tissue prolapse.

### DETAILED DESCRIPTION OF THE PREFFERED EMBODIMENT

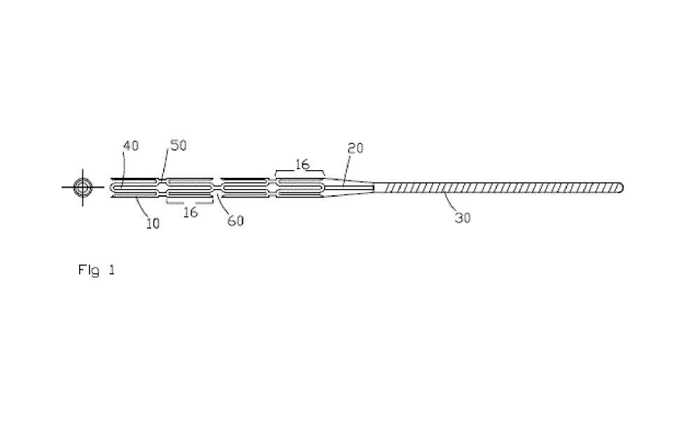

FIG. 1 shows the lumen occluding stent (10) made from a slotted tube with a guide wire (30) attached to its distal end. The tube can be solid or open shape made of elongated members. Although slots are preferred other shapes are contemplated including scoring the tubular wall so that expansion of the wall dimension meets less resistance and could also open along the score lines. A typical diameter of the stent portion is about 1.5 mm and a typical guide wire diameter shall be 0.035 inch. The guide wire shall be flexible and it can be made from stainless steel, cobalt chromium steel, or nickel containing steel. The guide wire can also be provided with a core wire at its center (not shown). The guide wire is attached to the stent portion by various attachment means such as adhesive bonding or welding. The guide wire can also be an integral part of the stent struts (40). The stent (10) is attached to the guide wire (30) by four to six struts (20). Struts (20) are a continuation of the stent (10). The stent described herein can be made by laser cutting, chemical etching or other machining means. The stent (10) of the shape and form can also be made from round or rectangular wire material. Wire material can be folded into sinusoidal form and the ends thereof can be welded to form the “sinusoidal rings” or “undulations” (16). These sinusoidal rings (undulations) are connected to each other by the struts (50). The struts (50) can be straight or may contain a varying tangent and thereby giving it a curvature. The lumen occluding stent has openings (60) cut out or scored to make the stent expandable and flexible. The design of the nature of the openings (60) will alter the radial strength and the flexibility of the stent. When more openings are designed into the slotted tube stent, can be straight slots or slots with curvatures, and less metal content will be left in the stent and hence will have less radial strength. When less openings are designed into the stent, the stent will be more rigid and will have more radial strength but less flexible. Depending on the end use application of the occluding stent, proper balance of the openings or scores and strut mass can be made to achieve the desired result.

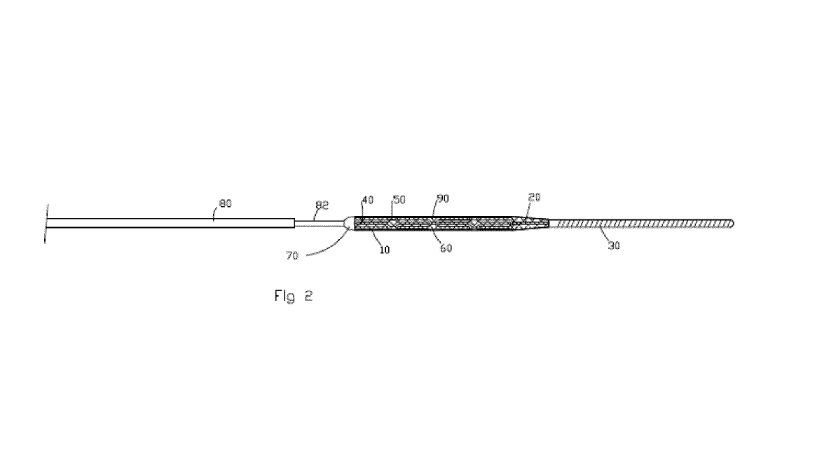

In FIG. 2, the slotted tube (10) is encased at least in part in a sheath of expandable material (90). When the stent is deployed to a lumen that will be occluded, the slotted tube is expanded using a balloon (70) in FIG. 2. The expanding material remains attached to the stent struts and forms a wall across the lumen and hence completely occluding the lumen. The stent struts act as a scaffolding support for the expanding material wall of the expanded material. The sheath 90 can lay over the distal end of the tube 10 or over the distal end and some of the side wall of the tube 10 leaving some or none of the side wall of the tube 10 uncovered. In this case the sheath material is over the stent body and not encasing the struts. On expanding the stent the sheath will be pushed against the lumen wall, sealing the lumen.

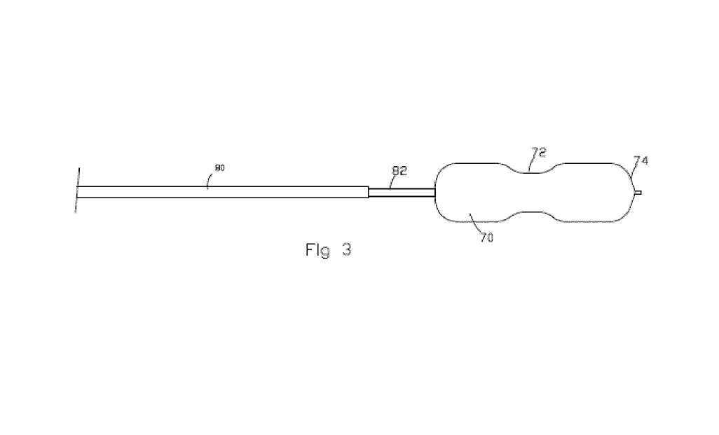

FIG. 2 shows the lumen occluding stent (10) which has been crimped onto a balloon catheter (80) which is similar to an angioplasty balloon. The balloon catheter has a shaft (80), with sufficient stiffness that will allow the operator to push and advance the catheter into the lumen that will be occluded, yet has a portion (82) proximal to the balloon that is flexible enough to give it track-ability characteristics enabling the advancement of the occluding device into the lumen. The balloon (70) itself can be made from any of the usual material used for angioplasty balloons, such as nylon, Polyethylene terapthalate (PET), polyethylene, and other materials that are able to form into a balloon that is relatively strong and has low distensibility characteristics. FIG. 3 shows a shape of the balloon catheter (80) having a balloon (70) which has a waist region (72). The balloon catheter may also provide a neck region (82) proximal to the balloon for added flexibility and for improved track-ability. The balloon waist described herein defines an area which has a diameter less than the diameter of the rest of the balloon in general. When the lumen occluding stent (10) is crimped on to the balloon (70) previously described having a neck region (82) and delivered into the lumen that is to be occluded. The deployed occluding stent takes the general shape of the balloon which has a neck region (82). Thereby imparting a neck region in the occluding stent, and which helps to anchor the stents in the body lumen.

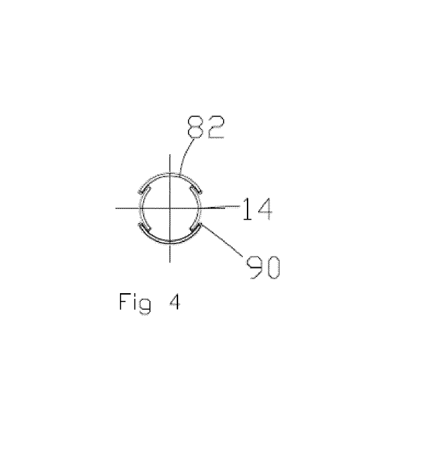

FIG. 4 shows an alternate method of folding the encased slotted tube stent (10) where the two opposite undulations (12) are crimped first and the orthogonal undulations (14) are crimped next so that one set of undulations overlaps the other set of undulations. By this means the degree of expansion of the encased material is much less and therefore will produce an expanded lumen occluding stent with less recoil. This is because the material undergoes lesser degree of deformation and as a result, due its larger initial perimeter and therefore is subjected to less strain and hence less recoil.

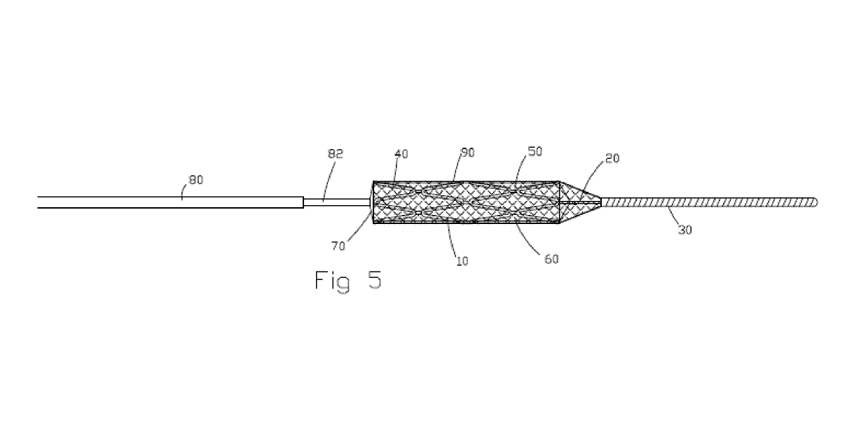

FIG. 5 shows a deployed (in its expanded state) lumen occluding stent (10) with the inflating balloon (70) within it. The struts of the stent are encased within the encasing material (90) and are impervious to fluids including blood or semen. Typically the encasing material (90) may encase only the most distal undulations of the lumen occluding stent.

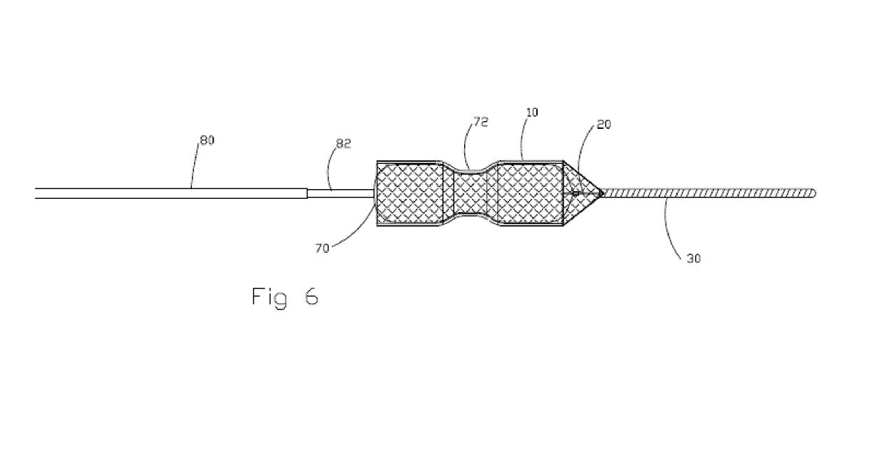

FIG. 6 shows a lumen occluding stent expanded by a delivery catheter balloon having a waist region (72), which has a diameter less than the rest of the balloon. When the stent is delivered into the lumen with such a balloon, the stent takes the general shape of the balloon. The balloon instead of having a waist may contain a bulge or a larger diameter, having an egg shape. Once the delivery catheter (80) is removed the stent shape remains allowing the tissue to prolapse into the stent and hence anchoring the stent into place. This will prevent the lumen occluding stent (10) from migrating from the position it was placed.

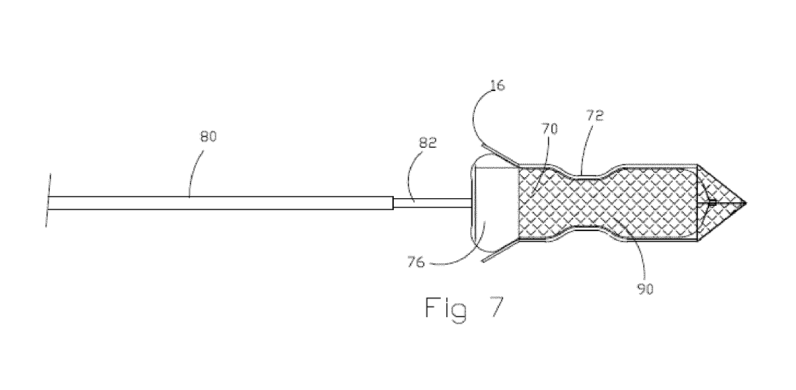

FIG. 7 shows another type of inflating balloon (70) having a larger diameter (76) at its proximal end. When a lumen occluding stent is expanded using such a balloon, the proximal end (16) of the stent will expand generally to match the balloon. This type of delivery balloon (70) will be useful when the lumen occluding stent is placed at the entrance to a lumen such as a blood vessel arising from the aorta or the fallopian tube arising from the uterus. When such expansion occurs, the stent (10) will not migrate further into the lumen because of its expanded proximal end.

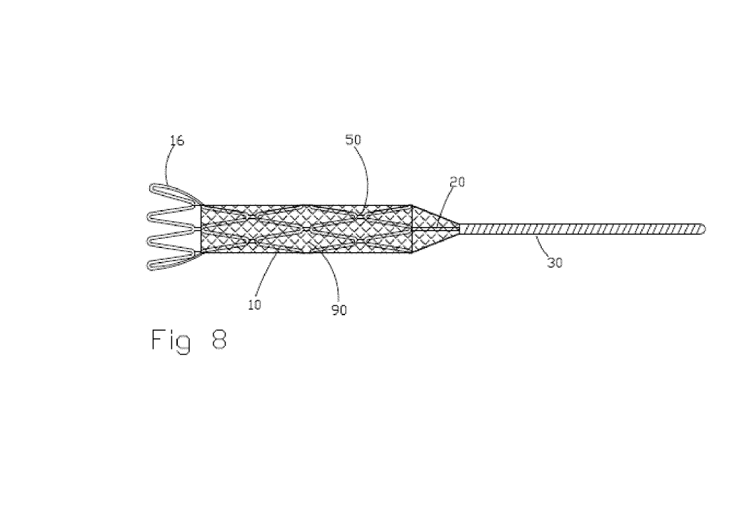

FIG. 8 shows a lumen occluding stent (10) with the proximal struts (16) expanded with a balloon described in FIG. 7. The encapsulating sheath (90) generally covers the entire stent except the expanding strut region (16). This feature will enable the lumen occluding stent to anchor into the tissue. Alternatively the encapsulating sheath (90) may cover only the most distal undulations of the lumen occluding stent and the adjacent cone or hemispherical region of the occluding stent.

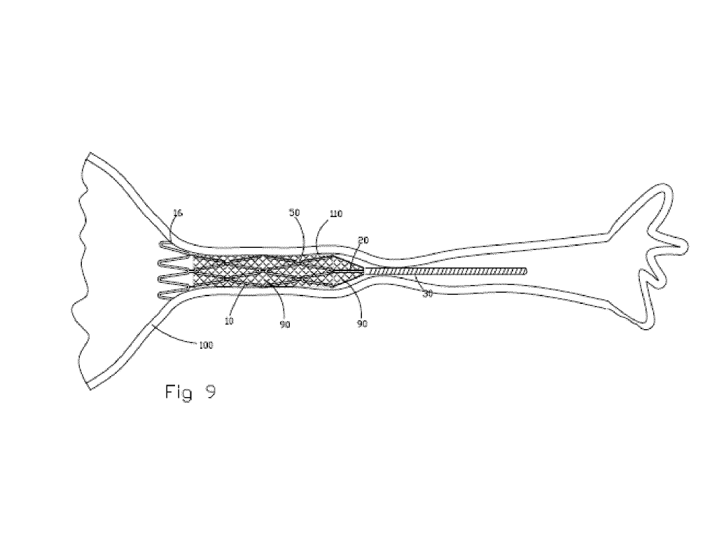

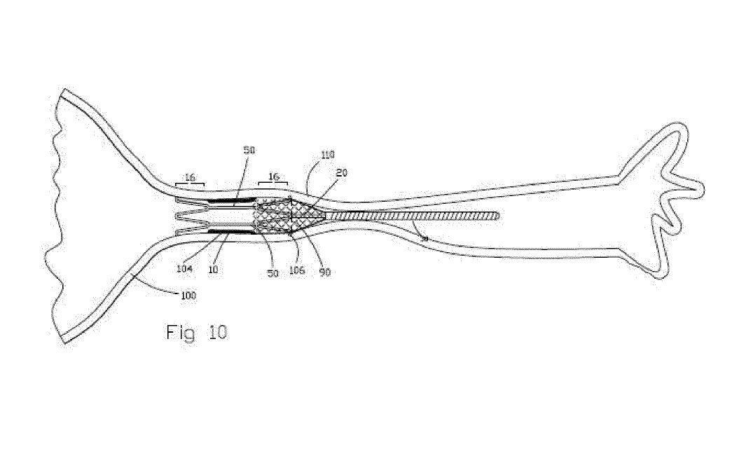

FIG. 9 shows the described lumen occluding stent (10) in FIG. 8 within the fallopian tubes of the human female. The expanding struts (16) are in the uterus and the occluding stent is within the fallopian tube. The sheath (90) encasing the lumen occluding stent is pressed against the inner wall of the fallopian tube in the stent region and encases the conical region, thereby completely occluding the lumen and forms a hermetic seal completely occluding the lumen immediately after the lumen occluding stent is deployed into the body lumen. FIG. 10, shows a similar lumen occluding stent, having only two undulations (16) connected by a longer connecting struts (50). The connecting struts are generally 1-5 mm long. The proximal undulation (16) is not encased in a sheath, but the second more distal undulation (16) and the adjacent cone (20) area are encased with the encasing material (90). The advantage of this lumen occluding stent is, once the stent is delivered, the inner wall tissue (104) can prolapse in between the longitudinal struts (50) and thereby locking and anchoring the stent in place, which will prevent the stent from migrating after placement. It is also possible to provide more than one undulation in the proximal region as well as at the distal region of the lumen occluding stent (10). The number of undulations used will essentially depend of the application, depending whether sufficient lumen length is available to place a longer lumen occluding stent and other criteria related to the lumen to be occluded.

I claim:

1. A lumen occluding assembly, comprising:
an expandable tubular body having proximal and distal ends defining a passage therethrough and having a smaller dimension for delivery and a larger dimension for contact of the surrounding lumen;
a sheath covering said passage so as to occlude the lumen when said body is at said larger dimension.

2. The assembly of claim 1, wherein:
said expandable tubular body having an outer wall and said sheath extends over at least a portion of said outer wall so that said sheath contacts the lumen in the larger dimension of said expandable tubular body.

3. The assembly of claim 1, wherein:
said expandable tubular body is formed having scores or openings when in said smaller dimension.

4. The assembly of claim 3, wherein:
said scores open to form openings when said expandable tubular body is in said larger dimension.

5. The assembly of claim 1, wherein:
said expandable tubular body is brought from said smaller to said larger dimension with an inflatable member selectively located within said passage.

6. The assembly of claim 5, wherein:
said inflatable member is removably mounted in said passage and secured to a distal end of a delivery member, said delivery member having a neck adjacent said inflatable member.

7. The assembly of claim 5, wherein:
said expandable tubular body further comprises adjacent a distal end thereof a plurality of spaced generally axially extending struts supporting an axially extending flexible guide wire.

8. The assembly of claim 7, wherein:
said sheath covers said struts.

9. The assembly of claim 2, wherein:
said sheath is located over said distal end of said expandable tubular body.

10. The assembly of claim 1, wherein:
said expandable tubular body further comprising opposed proximal and distal sinusoidal elongated member rings connected by a plurality of circumferentially spaced struts, said struts defining open areas therebetween for fixation by prolapsed of tissue defining an inner wall of the lumen.

11. The assembly of claim 10, wherein:
said sheath is mounted over said distal sinusoidal ring.

12. The assembly of claim 5, wherein:
said inflatable member has a larger proximal dimension when inflated so that said proximal end of said expandable tubular body in said larger dimension of said expandable tubular body has a larger proximal end dimension than the remainder of said expandable tubular body.

13. The assembly of claim 5, wherein:
said inflatable member has a smaller or larger dimension when inflated between a proximal and distal end thereof so that upon inflation said proximal and distal ends of said expandable tubular body have a larger or smaller peripheral dimension than another portion between said proximal and distal ends of said expandable tubular body.

14. The assembly of claim 5, wherein:
said expandable tubular body is formed from sinusoidally wound elongated members forming attached axially stacked and connected rings, said rings expandable to different peripheral dimensions between proximal and distal ends of said body to allow said body to be fixated with respect to a uterus at said proximal end and a fallopian tube.

15. The assembly of claim 3, wherein:
said sheath covers at least some of said openings.

16. The assembly of claim 1, wherein:
said lumen is occluded when said body reaches said larger dimension placing said sheath in contact with the lumen.

17. The assembly of claim 10, wherein:
said sinusoidal elongated member rings are disposed in said smaller dimension by layering of a first oppositely disposed bend pair over a second bend pair circumferentially offset from said first bend pair.

18. The assembly of claim 14, wherein:
said sinusoidal elongated member rings are disposed in said smaller dimension by layering of a first oppositely disposed bend pair over a second bend pair circumferentially offset from said first bend pair.

19. The assembly of claim 8, wherein:
said expandable tubular body having an outer wall and said sheath extends over at least a portion of said outer wall so that said sheath contacts the lumen in the larger dimension of said expandable tubular body;
said lumen is occluded when said body reaches said larger dimension placing said sheath in contact with the lumen.

20. The assembly of claim 1, wherein:
said expandable tubular body further comprises generally radially extending prongs to engage the lumen is said larger dimension for fixation of said expandable tubular body;
said prongs disposed adjacent said proximal end of said expandable tubular body and extending in a direction away from said distal end
